(19) United States
(12) Patent Application Publication
(10) Pub. No.: US 2008/0086096 A1
(43) Pub. Date: Apr. 10, 2008

(54) NANO PARTICLE ADDITIVES FOR VENOUS ACCESS CATHETER

(76) Inventors: Alexander Petrovich Voznyakovski, Saint Petersburg (RU); Leonid Malinin, Newton, MA (US); Pavel Yurievich Koblents, Saint Petersburg (RU); Vasiliy Gennadievich Abashkin, Saint Petersburg (RU); Paul DiCarlo, Middleboro, MA (US); Barbara Bell, Sudbury, MA (US); Christopher J. Elliott, Hopkinton, MA (US); Raymond Lareau, Westford, MA (US)

Correspondence Address: FAY KAPLUN & MARCIN, LLP, 15O BROADWAY, SUITE 702, NEW YORK, NY, 10038, US

(21) Appl. No.: 11/776,135

(22) Filed: Jul. 11, 2007

Related U.S. Application Data
(60) Provisional application No. 60/849,712, filed on Oct. 5, 2006.

(51) Int. Cl. A61M 5/32; C08J 5/02

(52) U.S. Cl. 604/265; 523/121

(57) ABSTRACT

Described is a catheter which includes a reinforced polymer shaft. The polymer includes nano particles having a dimension of less than about 6 nm. Described is also a method of forming a medical device. Granules of a selected polymer are prepared. Reinforced granules of the selected polymer are prepared. The reinforced granules include nano particles. The granules and the reinforced granules are then mixed in a ratio selected to obtain a desired concentration of the nano particles. The granules and the reinforced granules are extruded to form a substantially tubular element.

### PRIORITY CLAIM

This application claims the priority to the U.S. Provisional Application Ser. No. 60/849,712, entitled “Nano Particle Additives for Venous Access Catheter,” filed Oct. 5, 2006. The specification of the above-identified application is incorporated herewith by reference.

### BACKGROUND INFORMATION

The treatment of chronic disease often requires repeated and prolonged access to the vascular system for, e.g., the administration of medications, blood products, nutrients and other fluids and/or to withdraw blood. To avoid the discomfort and other side effects associated with the repeated insertion and removal of a needle for each session, a semi-permanent catheter, (e.g., a peripherally inserted central catheter (PICC)), may be used. As would be understood by those skilled in the art, a PICC is a catheter that is inserted into a vein at a peripheral location, such as the arm or leg, and threaded through the vein to the chest in proximity to the heart.

To simplify insertion and reduce discomfort, PICCs and other semi-permanent catheters are generally made thin and flexible limiting their structural strength. This, in turn, limits the maximum pressure and flow rate the catheter can support. Fluids escaping a catheter which has failed (e.g., after the maximum flow rate and/or pressure has been exceeded) may damage surrounding tissues.

In conjunction with the injection and/or withdrawal of fluids through such a semi-permanent catheter, it is often desirable to inject a contrast media to enhance visualization procedures. Typically, contrast media is introduced using a separate catheter designed to withstand the high injection pressures and flow rates necessary to disperse the media throughout an area of interest—pressures and flow rates which are generally beyond that permissible for typical semi-permanent catheters.

### SUMMARY OF THE INVENTION

In one aspect, the present invention is directed to a catheter comprising a reinforced polymer shaft, the polymer including nano particles having a dimension of less than about 6 nm.

In another aspect, the present invention is directed to a method of forming a medical device comprising preparing granules of a selected polymer, preparing reinforced granules of the selected polymer, wherein the reinforced granules include nano particles. The granules and the reinforced granules are then mixed in a ratio selected to obtain a desired concentration of the nano particles and the granules and the reinforced granules are extruded to form a substantially tubular element.

### BRIEF DESCRIPTION OF THE DRAWINGS

FIG. 1 is a cross sectional view showing an exemplary embodiment of a venous catheter comprising a nano particle reinforcement on the shell of the catheter;

FIG. 2 is a cross sectional view showing an exemplary embodiment of a venous catheter comprising a nano particle reinforcement throughout the catheter body;

FIG. 3 is a cross sectional view showing an exemplary embodiment of a venous catheter comprising a nano particle reinforcement on the divider of the catheter body;

FIG. 4 is a cross sectional view showing an exemplary embodiment of a venous catheter showing a nano particle reinforcement on discreet sections of the catheter body;

FIG. 5 is a cross sectional view showing an exemplary embodiment of a venous catheter showing a nano particle reinforcement on a layer of the catheter body;

FIG. 6 is an exemplary representation of a nano additive according to the present invention containing nanodiamonds;

FIG. 7 is an exemplary representation of a nano additive according to the present invention containing Fullerenes;

FIG. 8 is an exemplary representation of a nano additive according to the present invention containing carbon fibers;

FIG. 9 is a graph showing dependance of stress on deformation for a polymer according to the invention containing nano diamonds;

FIG. 10 is a graph showing dependance of stress on deformation for a polymer according to the invention containing fullerene particles;

FIG. 11 is a graph showing dependance of stress on deformation for a polymer according to the invention containing nano carbon fibers;

FIG. 12 is a diagram showing an exemplary embodiment of extrusion equipment for manufacturing medical tubing according to the invention;

FIG. 13 is an exemplary representation of a nano additive according to the present invention containing atomic zeolites;

FIG. 14 is an exemplary representation of a nano additive according to the present invention containing tetrahedral zeolites; and

FIG. 15 is an exemplary representation of a nano additive according to the present invention containing crystal zeolites.

### DETAILED DESCRIPTION

The present invention may be further understood with reference to the following description and the appended drawings, wherein like elements are referred to with the same reference numerals. The invention relates to devices for introducing contrast media, preferably at high pressure at a high flow rate. Specifically, the devices according to the invention may be used to inject the contrast media using a PICC.

As would be understood by those skilled in the art, the pressure exerted by the fluid is a function of the flow rate, the viscosity and the cross-sectional flow area of the catheter, among other variables. Accordingly, limitations on the fluid pressure and/or flow rate are often specified for various types of catheters to ensure that the catheter will not be damaged during use.

The catheter according to the present invention, may be used for both central venous access and for the injection of contrast media, decreasing discomfort and the time and expense of procedures. The catheter according to this invention, e.g., a PICC, is at least partially reinforced to enhance its burst pressure and maximum flow rate to levels suitable for the introduction of contrast media without compromising kink resistance or increasing the cross sectional profile of the catheter, as compared to conventional PICC's.

For example, catheters according to the present invention will withstand flow rates of about 1 to 100 cc/sec and pressures of more than the approximately 40 to 1600 PSI typical of power injection devices. The increased strength of the catheters according to the invention results from a reinforcement additive which, In one embodiment, is included in both a shaft and an extension tube of the catheter to achieve uniform burst resistant properties throughout the catheter. Alternatively, only the shaft, hub or bifurcation may be reinforced.

According to the exemplary embodiments of the present invention, the material forming the catheter comprises a polymeric matrix substance into which are dispersed one or more types of nano-size and micro-size additives, nano-size additives having dimensions of less than about 6 nm. For example the additives may include types of carbon such as ultra-dispersed diamonds (also referred to as nanodiamonds), carbon fibers and fullerenes. Fullerenes may comprise different geometries of carbon allotropes, examples of which include cylindrical carbon nanotubes and spherical buckyballs.

Introducing additives such as nanofibers, nanotubes and fullerenes to polymers has the potential of significantly enhancing mechanical, chemical and other properties of the material and, consequently, of devices made from such materials. For example, improved catheters may be manufactured from these enhanced materials. Fullerene (C₆₀) is capable of forming stable chemical bonds with a polymer material matrix. In one form it is a stable spherical molecule with a radius of about 0.357 nm. One of its characteristics is the ability to adjoin up to 26 —OH groups.

Nanotubes are another form of carbon additive that may be used according to the invention. Nanotubes have high mechanical strength and provide a large contact surface area with the polymeric matrix material, improving mechanical properties of the composite material, compared to those of the base polymer. Two additives have been developed by Zyvex Corp. in the USA especially for use in polyurethane matrices. The additives comprise both single walled nanotubes and multi-walled nanotubes.

Another additive according to embodiments of the invention comprises ultra-dispersed diamonds (UDD) or nano diamonds. These elements exist at the boundary between being a bulk state of the material and being individual molecules. UDD are synthetic diamonds that may be produced, for example, using a detonation method where the material is formed by explosive energy applied to carbon contained within explosive molecules. UDD is a product that combines properties that are generally incompatible and which are generally associated with different forms of carbon. The product has a core with a diamond structure displaying the hardness and chemical inertia of a diamond. In addition, UDD is a particle with nano dimensions, on the order of 4-6 nm, a rounded shape and a developed, active surface.

UDD are available from a variety of sources, such as the Diamond Center in Russia, the Chevron Texaco Technology Center in the USA, the International Technology Center also in the USA, and the NanoCarbon Research Institute in Japan. In an exemplary embodiment of the present invention, the usage of UDD as a filler with a polymer matrix for a material to make catheters is in the range of about 0.1% to 0.5% by mass.

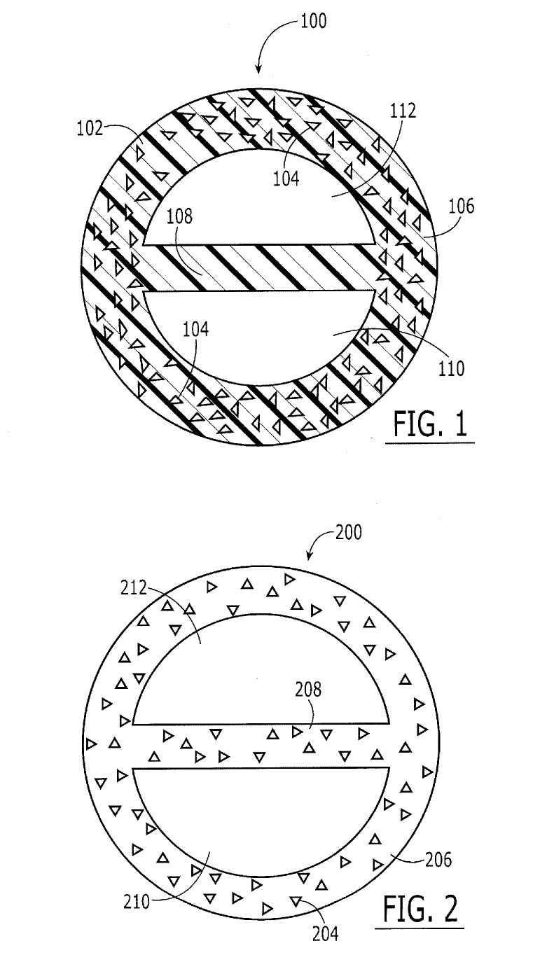

FIG. 1 shows an exemplary embodiment of a catheter in accord with the present invention. The exemplary catheter 100 is a dual lumen catheter in which the lumens 110, 112 are separated by a partition 108 extending along a longitudinal axis of the catheter 100. For example, the dual lumens 110, 112 may be used as supply and return passages for fluid circulating to and from the vascular system. To further improve the pressure resistance and ultimate strength of the base catheter material, nano and/or micro particles are added to the compound forming the catheter wall. The nano particles 104 may comprise one or more of the carbon compounds described above, mixed with a matrix material 102 such as a polymer. As would be understood by those of ordinary skill in the art, the present invention may also be used with multi-lumen catheters.

It will be apparent to those of skill in the art that the methods described herein to increase the strength of the wall of a catheter shaft may be applied selectively to any selected portions of the catheter or to the entire catheter to obtain properties desired for a particular application. Furthermore, the distribution of nano particles 104 may be varied both radially and longitudinally along the catheter 100 to obtain a desired variation of mechanical properties along and/or around the device. For example, a more pliable section of the catheter may be formed by locally reducing a concentration of nano particles 104 within the matrix material 102 while one or more regions of increased mechanical strength may be created by increasing the concentration of nano particles 104 in the region(s). In addition, portions of the catheter 100 subject to lesser forces (e.g., the divider 108) may be made without additives while portions subject to greater stresses (e.g., the shell 106) include the additive. The process of laminating different layers of nano particles may be used as well.

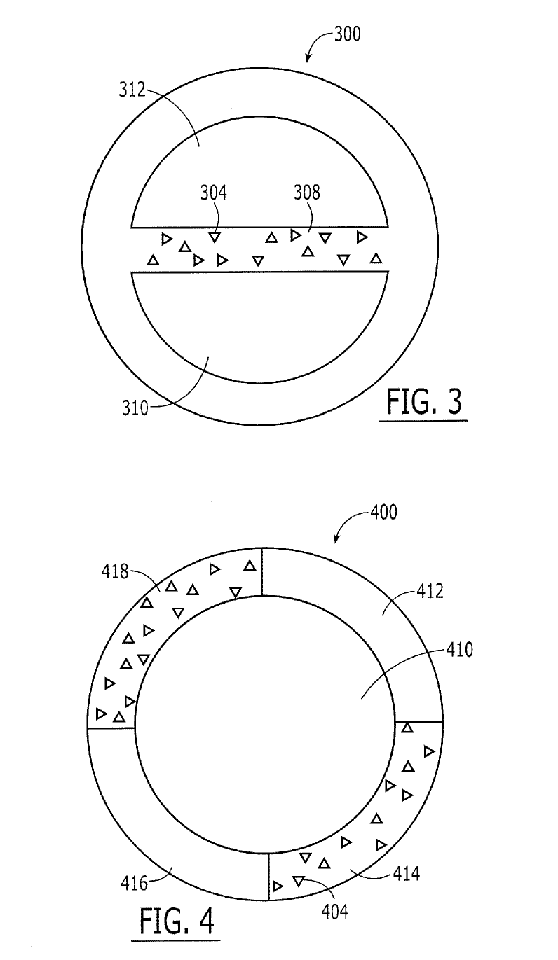

FIG. 2 shows another exemplary embodiment of a catheter in accord with the present invention. The exemplary catheter 200 is a dual lumen catheter in which the dual lumens 210 and 212 are separated by a partition 208 extending along a longitudinal axis of the catheter 200. Nano particles 204 are distributed throughout the catheter 200, including the partition 208 and the catheter shell 206. FIG. 3 shows an alternative embodiment of a dual lumen catheter 300 with lumens 310 and 312 separated by partition 308. Nano particles 304, however, are only distributed through the divider 308 in order to provide the partition 308 with reinforcement.

FIG. 4 shows another exemplary embodiment of a catheter 400 in accord with the present invention. Catheter 400 is comprised of a single lumen 410 and divided into quadrants 412, 414, 416, and 418. Nano particles 404 are distributed through quadrants 418 and 414. Although the nano particles 404 are shown to distributed through particular quadrants, it will be understood by those of ordinary skill in the art that the catheter may be divided into any number of discreet sections and the nano particles may be distributed through any or all of these sections.

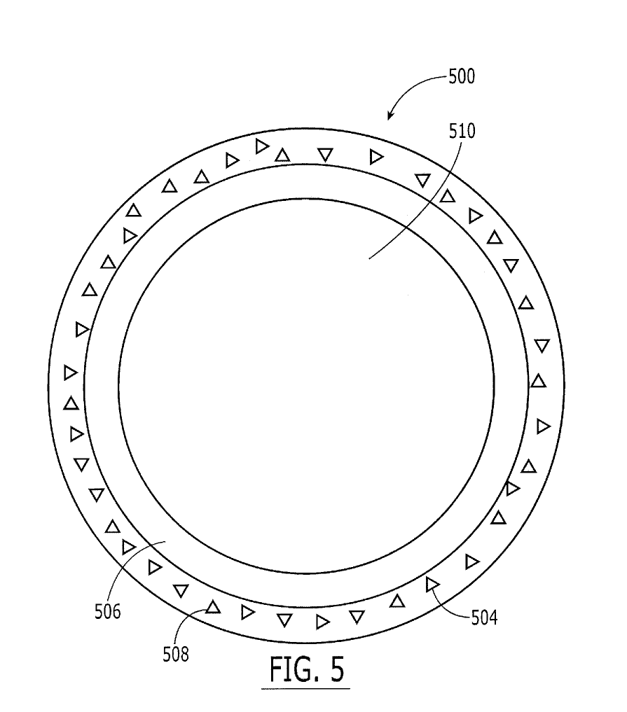

FIG. 5 shows another embodiment of a catheter in accord with the present invention. Catheter 500 is comprised of a single lumen 510, inner layer 506 and outer layer 508. The nano particles 504 are distributed through the outer layer 508. Although catheter 500 is shown with two layers, it will be apparent to one of ordinary skill in the art that the catheter may have multiple layers and that any or all of these layers may be reinforced with nano particles. In addition, although illustrated with a single lumen, the catheter may be comprised of multiple lumens.

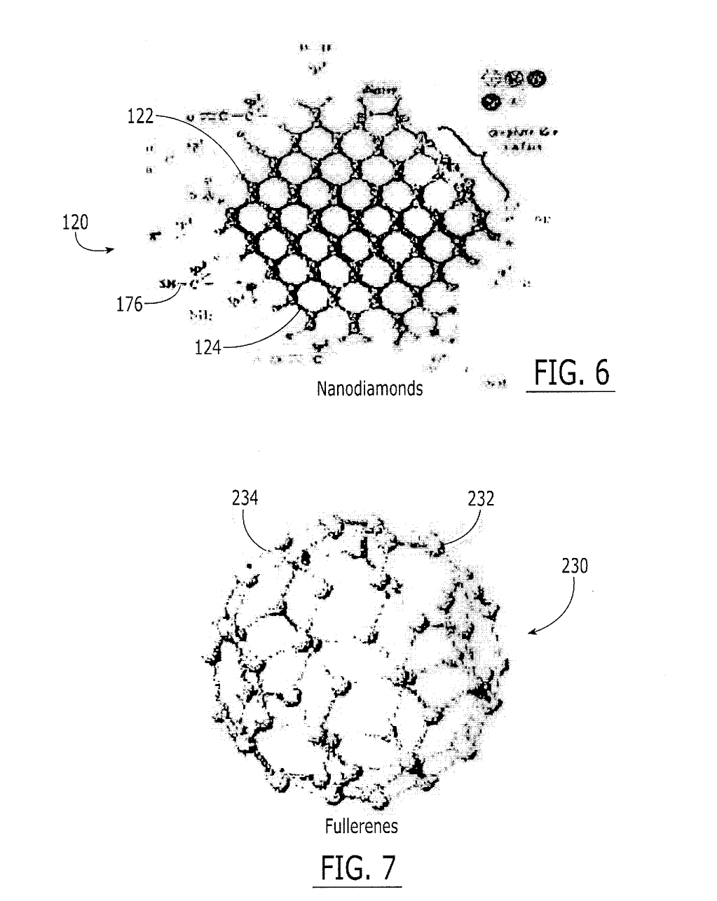

As described above, ultra-dispersed diamonds are one additive that may be used according to the invention to enhance the properties of the polymer forming the catheter wall. FIG. 6 shows a schematic representation of a UDD according to the invention. The UDD 120 comprises a plurality of carbon atoms 122 held in a crystalline lattice by bonds represented by lines 124. Additional compounds 126 may be attached to outer edges of the UDD molecule, giving it additional utility.

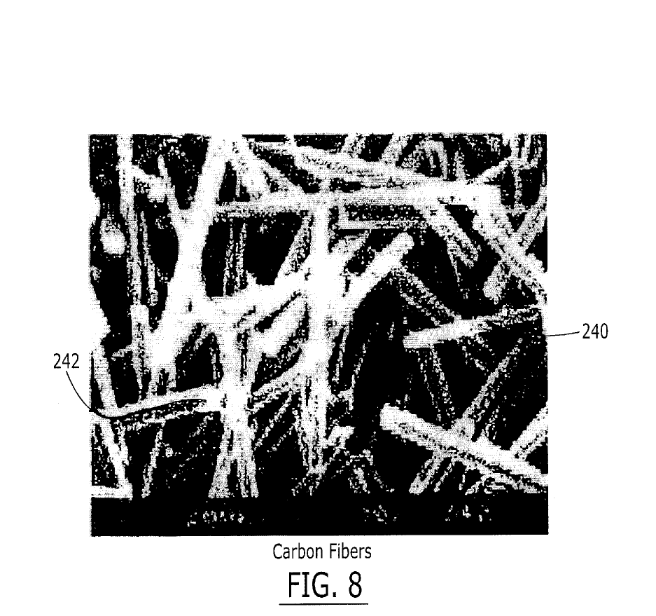

FIG. 7 shows of a fullerene C₆₀ additive for use in conjunction with an embodiment of the invention. A C₆₀ fullerene molecule 230 comprises 60 carbon atoms 232 connected into a stable spherical lattice by bonds 234. A C₆₀ fullerene molecule has a radius of 0.357 nm, and can adjoin up to 26 —OH groups. FIG. 8 shows exemplary carbon fibers 240 produced by processing plants or artificial polymer fibers which are characterized by a large, chemically active specific surface area 242 that gives this additive desirable chemical properties.

Testing of various nano particle additives was carried out to determine the improvement they bring over the baseline catheter material. The exemplary material comprised a polymer of polycarbonate-based polyurethane (PC-3585A) to which were added individual nano particle additives. Specifically, ultra-dispersed diamonds produced by detonation synthesis method, fullerenes of the spherical C₆₀ type and carbon fibers were added to the polymer. The tensile strength versus deformation for the base polymer and for the polymer with additives was measured for various concentrations of the additives.

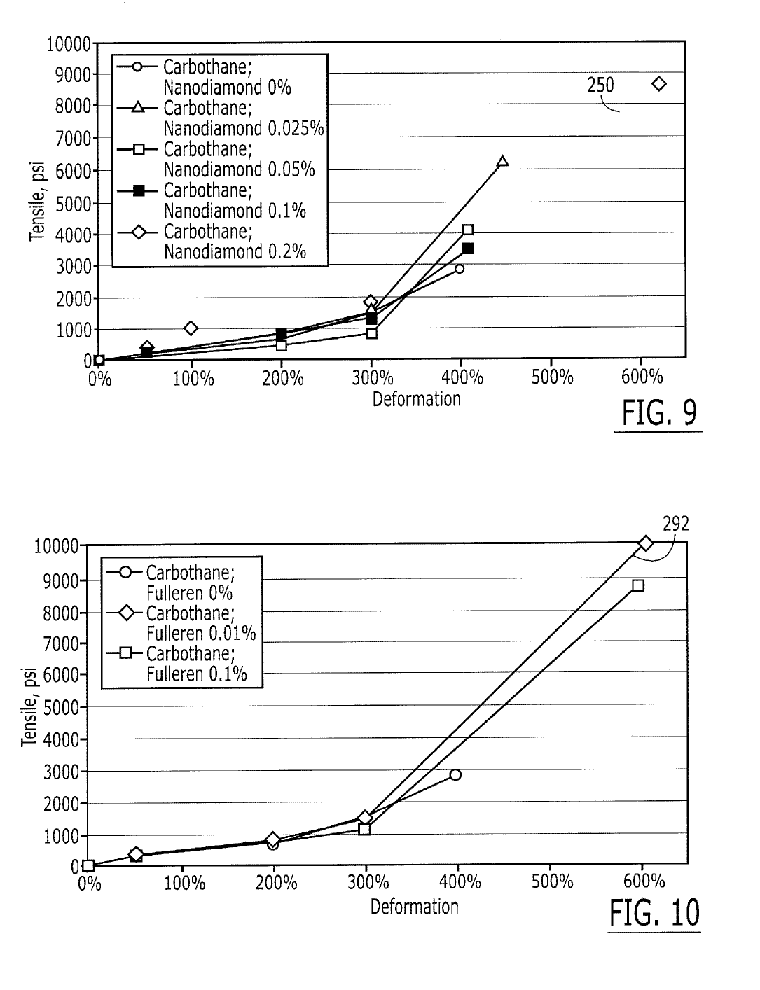

FIG. 9 shows curves of tensile strength and deformation for polycarbonate-based polyurethane with the addition of Nanodiamonds in percentages from 0% to 0.2% by weight. As can be seen in the graph, line 250 represents the performance of the Polycarbonate-based polyurethane matrix polymeric material with the addition of 0.2% by weight of UDD nano particles. The tensile strength of the material with additive increased by about threefold over the base material alone.

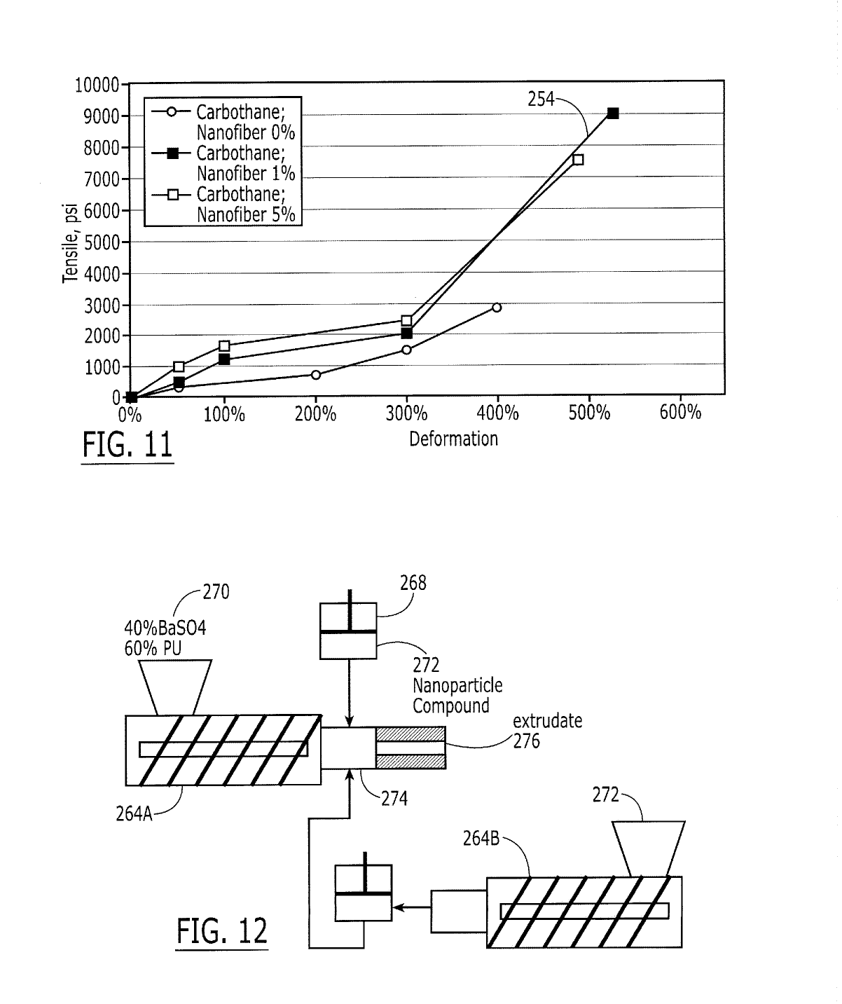

FIG. 10 shows a graph of tensile strength versus deformation for the polycarbonate-based polyurethane matrix polymer and an additive containing fullerene C₆₀. In this case, the additive ranged from 0% to 0.1% by weight. The tensile strength of the resulting polymer increased by threefold over the base material with the addition of 0.01% by weight of fullerene, as shown by line 252. Similar results were obtained by adding carbon fibers to the polycarbonate-based polyurethane matrix, as shown in FIG. 11. The carbon fibers are added in percentages between 0% and 5% by weight. As shown by line 254, the addition of 1% by weight of carbon fibers results in a threefold increase in the tensile strength of the material.

Those of skill in the art will understand that, for each of the nano particles to be added, there is an optimal concentration achieving a maximum increase in strength. Mixing an appropriate amount of nano particles to the base polymeric material results in catheter able to withstand higher pressures and/or flow rates reducing the duration of procedures and enabling the use of a catheter for both fluid injection and withdrawal as well for high pressures to, for example, achieve a desired dispersal of a contrast agent. The percentage of nano particles to base polymer is dependant on the base polymer's ability or tolerance to integrate the particles into its matrix without compromising bulk performance.

Although the preceding discussion related to nano particles of carbon used as additives, those of skill in the art will understand that other nano particles may be used as well. For example, zeolites or chelates may be used to incorporate metal atoms into the polymer and give it a degree of radiopacity. More traditional elements may be used to achieve the desired radiopacity, such as barium sulfate (BaSO4), bismuth salts, tungsten, etc. These may be specially processed to reduce their particle size to the nano level, for example by cryogenic grinding.

As would be understood by those skilled in the art, the matrix polymer used to form the base material of the catheter may be any of of several polymers in addition to the polycarbonate-based polyurethane described above. For example, thermoplastic polyurethane such as polyester, polyether, polycarbonate and polysiloxane based polymers may be used to form the structure of the catheter. Polyamides such as polyamide 12, polyamide 11, nylon and polyamide 6-12 may be used in other embodiments of the invention. In yet other embodiments, polyether block amide elastomers such as Pebax may be used as the matrix material, or polyolefins that include EVA, HDPE, MDPE, LDPE, SBS and SIBS. It will be understood by those of skill in the art that the above polymers as well as others may be used to form the material according to the invention, either alone or in combinations.

For certain applications it may be beneficial to include more than one additive to the polymeric matrix. For example, functionalized polymers or additives may be applied to improve the interface between the polymer and the nano particles, increasing the strength of the material. Furthermore, as would be understood by those skilled in the art, combinations of multiple nano scale additives may be introduced in the matrix material to achieve certain desirable mechanical, thermal, chemical and biological properties in the same product. In certain embodiments, nano scale additives may be combined with macro scale and micro scale additives, to achieve synergistic effects that could not be obtained with only one of the additives.

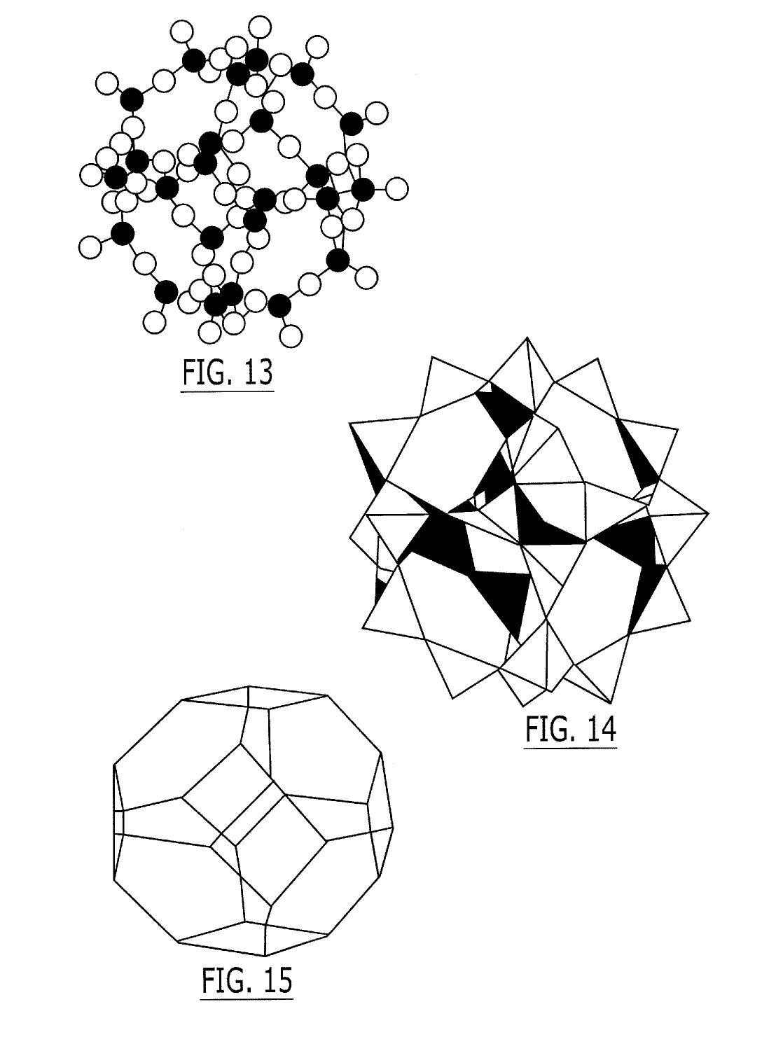

FIG. 13 shows a representation of an atomic zeolite nano particle that may be used as an additive to the matrix material to form a catheter. Other forms of the material are the tetrahedral zeolite, shown in FIG. 14 and the crystal zeolite shown in FIG. 15. Zeolites are a group of hydrated aluminosilicates of the alkali or alkaline earth metals, principally sodium, potassium, magnesium and calcium. Zeolites have three dimensional crystalline frameworks of tetrahedral silica or aluminum anions strongly bonded at all corners. The zeolite structures contain (—Si—O—Al—) linkages that form surface pores of substantially uniform diameter enclosing regular internal cavities and channels of discrete sizes and shapes, depending on the chemical composition and crystal structure of the specific zeolite involved.

The pore sizes of the various zeolite structures range from about 2 to about 4.3 Angstroms and the enclosed cavities may contain both metal cations and water molecules. The cations are loosely bound to the lattice and thus can carry out ion exchange. The water molecules in most zeolites can be reversibly driven out of the cavities. The structure of the zeolites allows them to perform various functions consistently within a broad range of chemical and physical environments. For example, they are able to selectively adsorb specific gas molecules and Zeolites have the ability to reversibly adsorb and desorb water without incurring chemical or physical changes in their matrix. They also have the ability to exchange cations in the crystal structure for other cations, such as lead, thallium, cesium or strontium, based on the specific exchange selectivity of the zeolite mineral.

During adsorption, gas molecules of different size are allowed to pass through the channels of the zeolite lattice. Depending on the size of the channels, the molecules are separated by size in a process known as molecular sieving. Each zeolite mineral has a distinctive ion exchange selectivity and capacity. Water molecules can pass through the channels and pores allowing cations present in the solution to be exchanged for cations in the structure. Several factors affects this process, such as solution strength, pH, temperature and the presence of other competing cations in the solution.

Chabazite and clinoptilolite are two exemplary minerals in the zeolite family that may be used as additives according to the invention. There are approximately 48 other materials in that family that may be considered. These compounds are the result of the chemical reaction between volcanic ash and alkaline water. Due to their high silica to aluminum ratio ranging from about 2:1 in chabazite to about 5:1 in clinoptilolite these minerals are stable and are less likely to dealuminate in acidic solutions than are synthetic zeolites.

The manufacture of polymeric material containing micro-particle additives presents some challenges. According to one embodiment of the invention, the manufacturing steps comprise producing a polymer with a high content of reinforcing nano particles, that will later be mixed with the pure base polymer. The high nano particle content polymer is manufactured by preparing a polymer solution in organic solvent. For example, in the case of polyurethane base materials, a tetrahydrofuran is prepared. Dissolving is effected by stirring in a capped bottle, for example at a temperature of about 45 C to 50 C. The amount of polymer that is dissolved may be, for example, about 10% to 18% of the solvent weight. In addition, traditional means such as twin screw and single screw extrusion may also be used to incorporate nano additives. Concentrates or direct incorporation may be applied as well.

The selected additives may be introduced into the solution during stirring. For example, UDD and carbon fiber additives are mixed as a dry material, and the fullerene C₆₀ is mixed as a solution. Afterwards, the solution is stirred for about 3 to 5 minutes at room temperature, and is poured either on a pre-leveled fluoroplastic substrate, or on a stretched cellophane film of, for example, approximately 1 mm thickness. The solution is dried for about 24 hours without heating. The films formed after cold drying are separated from the substrate and laid on a sheet of filter paper. The samples are then heated by an IR lamp for 5 to 10 hours or, alternatively, may be placed for 5 to 10 hours in a heating cabinet at a temperature of about 40° C. to 50° C. Granules of the polymer with the additive are collected after drying.

Pre-compounded material containing polymer and base additives or a direct combination of additive granules 270 are mixed and conveyed by a primary extruder 264A as the catheter is manufactured. FIG. 12 shows an exemplary extruder 260 in which one or more extruders 264A force the pure polymer 270 through a shaped die 274 together with an additive, in this case barium sulfate. One or more pistons 268 force an additional stream of polymer with additional nano particles 272 to mix with the stream 270 prior to or within the die, and to pass through the die 274. The piston reservoir displacing the additional polymer stream can be a simple reservoir or continuously fed by an additional screw extruder 264B or other traditional polymer or processing means such as an injection molding unit or ram extruder. By selecting the proper ratio of the extrusion rate of the streams of granules 270, 272 a desired concentration of one or more types of nano particles in the final extruded material 276 may be obtained.

The present invention has been described with reference to specific embodiments, and more specifically to a PICC catheter used for power injection of contrast media formed of a material containing carbon nano particles. However, other embodiments may be devised that are applicable to other medical devices and procedures, without departing from the scope of the invention. Those skilled in the art will understand that PICC's are not the only application for the materials containing nano particles according to the invention. For example, the reinforced material may be used to manufacture polymeric stents, drainage devices, infusion catheters for drug delivery, and/or polymeric stents for cardiology, peripheral, urinary and endoscopic applications. Additional embodiments of the material may be used for micro catheter delivery systems, stent delivery systems, low profile ports and PTCA/PTA devices. The catheters manufactured according tot he invention, in addition to PICC's may include regular catheters, CVC and venous access catheters. Accordingly, various modifications and changes may be made to the embodiments, particularly with regard to dimensions and materials, without departing from the broadest spirit and scope of the present invention as set forth in the claims that follow. The specification and drawings are accordingly to be regarded in an illustrative rather than restrictive illustrative rather than restrictive sense.

1. A catheter, comprising:
a reinforced polymer shaft, the polymer including nano particles having a dimension of less than about 6 nm.

2. The catheter of claim 1, wherein the nano particles include carbon nano particles.

3. The catheter of claim 1, wherein the nano particles comprise ultra-dispersed diamonds having dimensions of between about 4 nm and about 6 nm.

4. The catheter of claim 1, wherein the ultra-dispersed diamonds comprise about 0.2% by weight of the shaft.

5. The catheter of claim 1, wherein the nano particles comprise fullerene C₆₀ spheroid particles having a radius of about 0.357 nm.

6. The catheter of claim 1, wherein the fullerene C₆₀ comprises about 0.01% by weight of the shaft.

7. The catheter, wherein the nano particles comprise particles formed of carbon fibers.

8. The catheter of claim 7, wherein the carbon fibers comprise about 1% by weight of the shaft.

9. The catheter of claim 1, wherein the carbon nano particles comprise between about 0.1% and 0.5% by mass of the shaft.

10. The catheter of claim 1, wherein the nano particles comprise particles of zeolites.

11. The catheter of claim 10, wherein the zeolites comprise one of atomic, tetrahedral and crystal zeolites.

12. The catheter of claim 10, wherein the zeolites comprise one of chabazite and clinoptilolite.

13. The catheter of claim 1, wherein the polymer comprises one of thermoplastic polyurethane, polyamides, polyether block amide elastomers and polyolefins.

14. The catheter of claim 1, wherein the catheter is a peripherally inserted central catheter.

15. The catheter of claim 1, wherein the shaft is formed by extruding polymer granules of a base material with reinforced polymer granules containing the base material and a high concentration of the nano particles.

16. The catheter of claim 1, wherein the ratio of polymer granules of the base material and reinforced polymer granules is selected to obtain a desired concentration of the nano particles.

17. A method of forming a medical device, comprising:
preparing unreinforced granules of a selected polymer;
preparing reinforced granules of the selected polymer, the reinforced granules including nano particles; and
extruding the unreinforced granules with the reinforced granules to form a substantially tubular element.

18. The method of claim 17, further comprising:
preparing the reinforced granules by: dissolving the polymer in organic solvent, the polymer being between about 10% and 18% of the solvent weight; introducing in the solution a high concentration of the reinforcing additive; drying the solution of polymer and reinforcing additive to form a film; and recovering the reinforced granules from the dried solution.

19. The method of claim 17, further comprising:
dissolving the polymer at a temperature of between about 45° C. and 50° C.

20. The method of claim 17, further comprising:
introducing into the solution a reinforcing solution containing fullerene C₆₀.

21. The method of claim 17, further comprising:
introducing into the solution at least one of carbon fibers and ultra-dispersed diamonds in dry form.

22. The method of claim 17, further comprising:
preparing the reinforced granules containing at least one of zeolites and chelates nano particles.

23. The method of claim 17, further comprising:
drying the solution for about 24 hours without heating.

24. The method of claim 17, further comprising:
heating the drying solution for between about 5 and 10 hours to remove the solvent.

25. The method of claim 17, further comprising:
extruding the unreinforced granules and reinforced granules to form a shaft of a PICC.

26. The method of claim 17, wherein the desired concentration of nano particles is between about 0.01% and about 5%.

26. The method of claim 17, wherein a rate of extrusion of the unreinforced granules is controlled relative to a rate of extrusion of the reinforced granules to obtain a desired concentration of reinforced granules in the material of the device.

27. The method of claim 17, wherein the rate of extrusion of the reinforced granules relative to that of the unreinforced granules is varied over time to vary the concentration of reinforced granules in various parts of the device.

28. The method of claim 17, further comprising:
mixing the unreinforced granules and the reinforced granules in a ratio selected to obtain a desired concentration of the nano particles.

29. A catheter, comprising:
a reinforced polymer shaft, the polymer including nano particles having a dimension of less than about 6 nm.

30. A method of forming a medical device, comprising:
preparing granules of a selected polymer;
preparing reinforced granules of the selected polymer, the reinforced granules including nano particles;
mixing the granules and the reinforced granules in a ratio selected to obtain a desired concentration of the nano particles; and
extruding the granules and the reinforced granules to form a substantially tubular element.
